(19) United States
(12) Patent Application Publication
(10) Pub. No.: US 2009/0133437 A1
(43) Pub. Date: May 28, 2009

(54) Recovery of Co-Rich Product From a Mixed Gas Containing Heavy Hydrocarbons

(76) Inventors: Rian Reyneke, Katy, TX (US); Michael J. Foral, Aurora, IL (US)

Correspondence Address: NIXON & VANDERHYE, PC, 901 NORTH GLEBE ROAD, 11TH FLOOR, ARLINGTON, VA, 22203, US

(21) Appl. No.: 11/922,760

(22) PCT Filed: Jul. 28, 2005
(86) PCT No.: PCT/US2005/026657

§ 371 Date: Feb. 8, 2008

(51) Int. Cl. F25J 3/02; C01B 31/18; C01B 3/50; C07C 7/04

(52) U.S. Cl. 62/617; 62/618; 62/620

(57) ABSTRACT

A novel process is disclosed which produces a CO-rich stream from a stream containing hydrogen, carbon monoxide, methane, and components heavier than methane. The process utilizes a combined CO purification and demethanizer column (5) which reduces the overall capital cost of the process, and efficient heat integration which reduces the energy required by the process. This process is useful in recovering a CO-rich stream (10) from the effluent (1) of an autothermal cracking reactor. It is particularly useful when one or more of the heavy components (6) has a higher value as pure product than when admixed with methane (11), and when product of a purified hydrogen stream (8) is also desirable.

### STATEMENT REGARDING FEDERALLY SPONSORED RESEARCH OR DEVELOPMENT

This invention was made with government support under United States Department of Energy Cooperative Agreement No. DE-FC07-01ID 14090.

### PROCESS FOR THE PRODUCTION OF OLEFINS

The present invention relates to a process for the recovery of carbon monoxide, and optionally hydrogen, from a process stream comprising carbon monoxide, hydrogen, methane, and hydrocarbons heavier than methane (i.e. having two or more carbon atoms).

Carbon monoxide and hydrogen may be formed in a number of processes, including the endothermic steam reforming of hydrocarbons, particularly methane or naphtha-range hydrocarbons. The hydrogen and carbon monoxide may be obtained as a gas mixture comprising the hydrogen, carbon monoxide and, typically, methane, which can then be treated to recover separate hydrogen-rich and carbon monoxide-rich products. In commercial-scale operations cryogenic distillation is most often used to recover and purify the hydrogen and carbon monoxide products, though pressure swing adsorption and other techniques can also be employed.

Many cryogenic systems for the recovery of carbon monoxide and hydrogen from mixed gases utilize a methane wash tower in which the carbon monoxide and hydrogen-containing gas mixture (process stream) is contacted in a trayed or packed tower with a liquid methane-rich stream. Much of the carbon monoxide and methane contained in the gas mixture is dissolved into the methane-rich adsorbent to produce a bottoms liquid containing primarily carbon monoxide, methane, and dissolved hydrogen, and an overhead product containing primarily hydrogen and residual amounts of carbon monoxide and methane. The bottoms liquid is typically stripped of hydrogen in one or more stripping columns or vessels and the resulting stripped liquid is separated in a cryogenic carbon monoxide purification column into a (typically) gaseous carbon monoxide overhead product and a liquid methane bottoms product. Such processes are described, for example, in U.S. Pat. No. 4,888,035, U.S. Pat. No. 5,133,793, U.S. Pat. No. 5,592,831, U.S. Pat. No. 6,082,134, U.S. Pat. No. 6,269,657 and US 2002/0134243.

Other cryogenic systems for the recovery of carbon monoxide and hydrogen, which do not utilize a methane wash column but rely on partial condensation and stripping steps, are described, for example, in U.S. Pat. No. 5,509,271 and U.S. Pat. No. 6,173,585.

None of the above documents address the issues of recovery of carbon monoxide and hydrogen from a gas mixture comprising hydrocarbons heavier than methane (i.e. having 2 or more carbon atoms). Process streams comprising hydrocarbons with 2 or more carbons atoms may be obtained, for example, where carbon monoxide and hydrogen-containing gas mixtures arise from other sources, such as refinery off-gases and cracked gases produced during the production of light olefins via oxidative dehydrogenation. It is advantageous to remove at least some of these hydrocarbons with 2 or more carbons atoms from the process stream before it is charged to the carbon monoxide and hydrogen recovery process. The removal of the hydrocarbons with 2 or more carbons atoms from the cryogenic recovery process improves the energy efficiency of the process and can also reduce the risk of foaming in the cryogenic distillation system. In addition, the hydrocarbons with 2 or more carbons atoms themselves can be more valuable as a purified product than they would be as fuel stream, for example, if they were mixed with methane and burned.

U.S. Pat. No. 6,578,377 teaches a process for recovering hydrogen and carbon monoxide from a gas mixture containing carbon monoxide, hydrogen, methane, and hydrocarbons heavier than methane (hydrocarbons with 2 or more carbons atoms). The process comprises a partial condensation step for separating the gas mixture into a stream depleted in hydrocarbons with 2 or more carbons atoms and a reject stream enriched in hydrocarbons with 2 or more carbons atoms, sending the stream depleted in hydrocarbons with 2 or more carbons atoms into a methane wash tower, subjecting the methane-rich wash tower bottoms liquid to a hydrogen stripping step, and recovering a CO-enriched overhead product and a methane-enriched liquid bottoms product from the hydrogen-stripped liquid using a cryogenic distillation system, and using at least a portion of the methane-enriched liquid to provide the methane-rich reflux to the methane wash column.

The partial condensation method of U.S. Pat. No. 6,578,377 concentrates the hydrocarbons with 2 or more carbons atoms in the reject stream so that the molar ratio of hydrocarbons with 2 or more carbons atoms to methane in the gas phase is less than 0.05, preferably less than 0.02. However, this process has the disadvantage that such a process is relatively inefficient in separation of the carbon monoxide and hydrocarbons with 2 or more carbons atoms. Thus, as the temperature of the partial condensation decreases, the amount of hydrocarbons with 2 or more carbons atoms in the uncondensed vapor decreases, but also the recovery of carbon monoxide to the uncondensed vapor decreases.

The present invention provides an improved (i.e. more efficient) process for separating carbon monoxide from hydrocarbons with 2 or more carbons atoms in a mixed stream comprising carbon monoxide, hydrogen, methane, and hydrocarbons with 2 or more carbons atoms.

Thus, in a first aspect, the present invention provides a process for the recovery of a CO-rich stream comprising at least 50 mol % carbon monoxide and at least 2 mol % hydrogen from a mixed stream comprising carbon monoxide, hydrogen, methane and hydrocarbons with two or more carbon atoms, said process comprising the steps of:
a. passing the mixed stream to a first separation step to separate the mixed stream into one or more first intermediate streams comprising hydrocarbons with two or more carbon atoms and a reduced quantity of the carbon monoxide, hydrogen and methane relative to the mixed stream, and a second intermediate stream comprising carbon monoxide, hydrogen, methane and a reduced quantity of the hydrocarbons with 2 or more carbons atoms relative to the mixed stream,
b. passing the second intermediate stream to a second separation step to separate the second intermediate stream into one or more third intermediate streams comprising carbon monoxide and methane and a reduced quantity of hydrogen relative to the second intermediate stream, and a hydrogen-rich stream which comprises a reduced quantity of carbon monoxide and methane relative to the second intermediate stream, and
c. passing at least one of the one or more first intermediate streams and at least one of the one or more third intermediate streams to a third separation step to recover a CO-rich stream comprising at least 50 mol % carbon monoxide and at least 2 mol % hydrogen, a methane-rich stream, and a heavy hydrocarbon stream comprising hydrocarbons with 2 or more carbon atoms,
wherein the at least one of the one or more first intermediate streams and the at least one of the one or more third intermediate streams are passed to the third separation step without treatment to separate hydrogen.

The mixed stream comprising carbon monoxide, hydrogen, methane and hydrocarbons with two or more carbon atoms may be derived from any suitable source, such as from the product stream of an autothermal cracking process for the production of olefins. Such a process is described further below.

Typically, the mixed stream comprises carbon monoxide, hydrogen, methane and hydrocarbons with two or more carbon atoms in the following proportions:

| | |
|---|---|
| carbon monoxide: | 2 to 50 mol %, such as 5 to 40 mol %; |
| hydrogen: | 5 to 80 mol %, such as 20 to 70 mol %; |
| methane: | 5 to 60 mol %, such as 10 to 40 mol %; and |
| hydrocarbons with 2 or | 2 to 30 mol %, such as 2 to 20 mol %. |
| more carbon atoms: | |

Step (a) of the process of the present invention comprises passing the mixed stream to a first separation step to separate the mixed stream into one or more first intermediate streams comprising hydrocarbons with two or more carbon atoms and a reduced quantity of the carbon monoxide, hydrogen and methane relative to the mixed stream, and a second intermediate stream comprising carbon monoxide, hydrogen and methane and a reduced quantity of the hydrocarbons with 2 or more carbons atoms relative to the mixed stream.

The first separation step preferably comprises passing the mixed stream to a chilling train comprising one or more stages for partial condensation of the process stream, for example by cooling in a heat exchanger, followed by vapor/liquid separation in a flash drum or in a rectification column (in which the vapor is purified or enriched in carbon monoxide, hydrogen and methane by contact with a counterflowing stream of liquid condensed from the vapor).

Within each stage of the chilling train, the chilling and partial condensation are followed by liquid and vapor separation. The vapor is passed to the next chilling/partial condensation stage (if any). The uncondensed vapor stream from the final chilling/partial condensation stage forms the second intermediate stream comprising carbon monoxide, hydrogen, methane and a reduced quantity of hydrocarbons with 2 or more carbon atoms relative to the mixed stream.

The liquid stream removed from each partial condensation stage forms one or the one or more first intermediate streams.

Alternatively, the first separation step may comprise a combined rectification and chilling process, such as a dephlegmator, a distillation column with side condensers or an advanced heat integrated rectifier system, such as described in U.S. Pat. No. 6,343,487.

Step (a) of the process of the present invention produces one or more first intermediate streams comprising hydrocarbons with 2 or more carbons atoms and a reduced quantity of carbon monoxide, hydrogen and methane relative to the mixed stream and a second intermediate stream comprising carbon monoxide, hydrogen, methane and a reduced quantity of hydrocarbons with 2 or more carbon atoms.

The one or more first intermediate streams will typically comprise at least 5 mol % hydrocarbons with 2 or more carbons atoms.

The amount of separation required in step (a) to achieve this will depend on the actual amount of hydrocarbons with 2 or more carbons atoms in the process stream to step (a), but, in general, the first intermediate streams will (in total) typically comprise at least 75% by mole of the hydrocarbons with two or more carbon atoms that were present in the initial process stream, such as at least 90% by mole of the hydrocarbons with 2 or more carbons atoms. Higher percentages of the hydrocarbons with 2 or more carbons atoms in the first intermediate streams, and hence lower percentages in the second intermediate stream will be required where the process stream to step (a) comprises relatively more hydrocarbons with 2 or more carbons atoms, such that the absolute amount of hydrocarbons with 2 or more carbons atoms which remain in the second intermediate stream is maintained at a suitably low level. Typically, the second intermediate stream comprises less than 1% by mole of hydrocarbons with 2 or more carbon atoms.

Typically (i.e. using the temperatures required to achieve less than this percentage of hydrocarbons with two or more carbon atoms in the second intermediate stream) the one or more first intermediate streams will (in total) comprise at least 2% of the carbon monoxide that was present in the initial process stream, and at least 10%, usually at least 20%, of the methane that was present in the initial process stream. The first intermediate streams will (in total) also usually comprise less than 1% of the hydrogen that was present in the initial process stream.

In step (b) of the process of the present invention, the second intermediate stream comprising carbon monoxide, hydrogen, methane and a reduced quantity of hydrocarbons with 2 or more carbon atoms is passed to a second separation step to separate the stream into one or more third intermediate streams comprising carbon monoxide and methane, and a reduced quantity of hydrogen relative to the second intermediate stream, and a hydrogen-rich stream which comprises a reduced quantity of carbon monoxide and methane relative to the second intermediate stream.

The second separation step may comprise at least one stage of chilling and partial condensation of the second intermediate stream, preferably followed by rectification in a carbon monoxide rectification column, wherein the stream is contacted with a counter-flowing reflux liquid, such as a recycled carbon monoxide containing stream. The overhead stream from the rectification column can be partially condensed and separated in a reflux drum. The liquid from the reflux drum is directed as reflux liquid to the carbon monoxide rectification column.

In an alternate configuration, the overhead stream from the CO rectification column may be passed first to a reflux re-contacting drum which serves to chill and partially rectify the overhead stream from the rectification column by direct contact with cold reflux liquid. The overhead stream from the reflux re-contacting drum is then cooled and partially condensed and separated in a reflux drum to produce a net overhead vapor stream comprising hydrogen and a liquid stream which is directed to the reflux re-contacting drum. It should be noted that the reflux re-contacting drum discussed above may be combined with the rectification column into a single vessel.

Whether or not a reflux re-contacting drum is used, the net overhead vapor stream from the reflux drum forms the hydrogen-rich stream according to the present invention. The hydrogen-rich stream comprises primarily hydrogen, by which is meant at least 80 mol % hydrogen. Typically the hydrogen-rich stream contains less than 1 mol % methane.

The bottoms stream from the rectification column and, where present, a portion of the liquid stream from the reflux re-contacting drum form the one or more third intermediate streams comprising carbon monoxide and methane and a reduced quantity of hydrogen relative to the second intermediate stream.

In an alternative embodiment, a methane wash column may be used instead of a carbon monoxide rectification column.

Typically, the one or more third intermediate streams will comprise less than 5 mol % hydrocarbons with 2 or more carbon atoms and less than 5 mol % hydrogen.

In step (c) of the process of the present invention at least one of the one or more first intermediate streams and at least one of the one or more third intermediate streams are directed to a third separation step to recover a CO-rich stream comprising at least 50 mol % carbon monoxide and at least 2 mol % hydrogen, a methane-rich stream and a heavy hydrocarbon stream comprising hydrocarbons with 2 or more carbon atoms. Preferably, the CO-rich stream comprises at least 80 mol % carbon monoxide.

It is a feature of the present invention that the at least one of the one or more first intermediate streams and the at least one of the one or more third intermediate streams can be (and are) passed to the third separation step without treatment to separate hydrogen.

Since hydrogen separation steps, such as hydrogen stripping, generally also result in loss of carbon monoxide to the hydrogen product or vent streams, the process of this invention has the advantage that the amount of carbon monoxide passed to the third separation step, and hence capable of being recovered, is increased. The absence of hydrogen stripping steps also saves the cost associated with such equipment.

The third separation step preferably comprises a single distillation column which acts as a combined demethanizer/CO purification column, and from which is removed a gaseous overhead stream, a liquid sidestream and a liquid bottoms stream, as respectively said CO-rich stream, said methane-rich stream and said heavy hydrocarbon stream

Suitably, each of the one or more third intermediate streams enters the distillation column at a location above that where the liquid sidestream is recovered, and each of the one or more first intermediate streams enters the distillation column at a location below that where the liquid sidestream is recovered.

The heavy hydrocarbon stream suitably comprises predominantly (at least 95 mol %, preferably at least 99 mol %) hydrocarbons with 2 or more carbon atoms and is substantially free of methane, hydrogen and carbon monoxide.

Typically the CO-rich stream comprises less than 40 mol % hydrogen, preferably less than 20 mol %. Typically the CO-rich stream comprises less than 1 mol % methane.

Optionally, the CO-rich stream may be treated to remove at least some of the hydrogen therein before further use. For example, the hydrogen in said CO-rich stream may be removed through adsorption onto a suitable adsorbent or through reaction with the carbon monoxide contained in said CO-rich stream to produce a purified carbon monoxide stream containing less than 1 mol % hydrogen.

In a further embodiment, at least a portion of said CO-rich stream may be used as at least a portion of the working fluid in a CO-rich vapor recompression refrigeration system, the CO-rich vapor recompression refrigeration system providing process chilling duty to one or more of the first, second, and/or third separation steps.

Typically, this use of a CO-rich vapor recompression refrigeration system may comprise the steps of:
i. Compressing at least a portion of said CO-rich stream to a relatively high pressure to form a relatively high-pressure CO-rich stream,
ii. Cooling and partially condensing at least a portion of the relatively high-pressure CO-rich stream of step (i) to produce a cooled and at least partially condensed working fluid stream,
iii. Reducing the pressure of the cooled and at least partially condensed working fluid stream of step (ii) to produce a reduced-pressure working fluid stream,
iv. Heating and at least partially vaporizing the reduced-pressure working fluid stream of step (iii) to provide the process chilling duty to one or more of the first, second, and/or third separation steps and produce a heated reduced pressure working fluid stream, and
v. Recompressing at least a portion of the heated reduced pressure working fluid stream in step (i)

The methane-rich stream suitably comprises primarily (at least 75 mol %, preferably at least 85 mol %) methane, with relatively low concentrations of hydrogen, carbon monoxide and hydrocarbons with 2 or more carbon atoms. Typically, the methane-rich stream comprises less than 10 mol % carbon monoxide, less than 5 mol % hydrocarbons with 2 or more carbon atoms and less than 1 mol % hydrogen.

The methane-rich stream may also be utilized in the CO-rich vapor recompression refrigeration system. For example, the methane-rich stream may be recovered as a liquid stream and at least a portion of said methane-rich stream reduced in pressure, warmed, and at least partially vaporized to provide at least a portion of the cooling duty required in step (ii) of the CO-rich vapor recompression refrigeration system.

The methane-rich stream may, after being optionally pumped, be cooled to a temperature at least 5° C. below its bubble point before it is reduced in pressure, warmed, and at least partially vaporized, said bubble point being defined at the pressure at which said methane-rich stream is recovered (prior to the optional pumping step).

In a further embodiment, at least a portion of said methane-rich stream may first be mixed with at least a portion of the hydrogen-rich stream from step (b) and the combined stream warmed and at least partially vaporized to provide at least a portion of the condensing duty needed in the CO-rich vapor recompression refrigeration system.

The process of the present invention is preferably operated at elevated pressures.

In particular, steps (a) and (b) are preferably performed at a pressure of at least 15 barg, for example, in the range 15-40 barg.

Step (c) is preferably performed at a pressure of at least 7 barg, more preferably at a pressure of at least 10 barg.

In a preferred embodiment of the present invention, the mixed stream comprising carbon monoxide, hydrogen, methane and hydrocarbons with 2 or more carbons atoms is derived from the product stream of an autothermal cracking process for the production of olefins.

Autothermal cracking is a route to olefins in which a hydrocarbon-containing feedstock is mixed with oxygen and passed over an autothermal cracking catalyst. The autothermal cracking catalyst is capable of supporting combustion beyond the fuel rich limit of flammability. Combustion is initiated on the catalyst surface and the heat required to raise the reactants to the process temperature and to carry out the endothermic cracking process is generated in situ. Generally the hydrocarbon-containing feedstock and the oxygen are passed over a supported catalyst capable of supporting combustion beyond the fuel rich limit of flammability to produce the olefin product. The autothermal cracking process is described in EP 332289B; EP-529793B; EP-A-0709446 and WO 00/14035.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

Autothermal cracking may be used to convert both liquid and gaseous hydrocarbons into olefins. Suitable liquid hydrocarbons include naphtha, gas oils, vacuum gas oils and mixtures thereof. Preferably, however, gaseous hydrocarbons such as ethane, propane, butane and mixtures thereof are employed.

The hydrocarbon-containing feedstock may be fed with any suitable oxygen-containing gas. Suitably, the oxygen-containing gas is molecular oxygen, air and/or mixtures thereof. The oxygen-containing gas may be mixed with an inert gas such as nitrogen or argon.

Preferably the hydrocarbon-containing feedstock and oxygen-containing gas are fed to the autothermal cracker at a ratio of hydrocarbon to oxygen-containing gas of 5 to 16 times, preferably 5 to 13.5 times, more preferably 6 to 10 times, the stoichiometric ratio of hydrocarbon to oxygen-containing gas required for complete combustion of the hydrocarbon to carbon dioxide and water.

The hydrocarbon is passed over the catalyst at a gas hourly space velocity of greater than 10,000 h⁻¹, preferably above 20,000 h⁻¹ and most preferably, greater than 100,000 h⁻¹. It will be understood, however, that the optimum gas hourly space velocity will depend upon the pressure and nature of the feed composition.

Additional feed components may be co-fed into the autothermal cracker, such as hydrogen, carbon monoxide, carbon dioxide or steam. Preferably, hydrogen is co-fed with the hydrocarbon-containing feedstock and oxygen-containing gas into the autothermal cracker. Suitably, the molar ratio of hydrogen to oxygen-containing gas is in the range 0.2 to 4. Hydrogen co-feeds are advantageous because, in the presence of the catalyst, the hydrogen combusts preferentially relative to the hydrocarbon, thereby increasing the olefin selectivity of the overall process.

The autothermal cracking process is suitably be carried out at a catalyst exit temperature in the range 600° C. to 1200° C., preferably, in the range 850° C. to 1050° C. and, most preferably, in the range 900° C. to 1000° C. To avoid further reactions taking place, the ATC product stream should be rapidly cooled, typically by cooling to between 750-600° C. within 20 milliseconds of formation. Advantageously wherein the autothermal cracking process is operated at a pressure of greater than 20 barg the products are cooled to between 750-600° C. within 10 milliseconds of formation.

The reaction products are quenched with water as they emerge from the autothermal cracker, typically in a suitable quench tower. The ATC product stream, in addition to olefins, typically comprises unreacted hydrocarbons, hydrogen, carbon monoxide, methane, and small amounts of acetylenes, aromatics and carbon dioxide, which need to be separated from the desired olefins.

Typically, carbon dioxide is removed from the ATC product stream first, for example, using an amine-based absorption system such as MEA or TEA (or mixtures of both), or other commercially available CO₂ removal process.

Any residual water may be removed next. Any suitable drying process may be used, for example by use of a suitable molecular sieve.

All or a portion of this ATC product stream (after carbon dioxide removal and after water removal) may then be passed as a mixed stream comprising carbon monoxide, hydrogen, methane and hydrocarbons with 2 or more carbons atoms, for treatment to recover a CO-rich stream as in the first aspect of the present invention.

In some embodiments it may be desirable to direct the ATC product stream to a front-end rectification column after the treatment to remove carbon dioxide and/or water and prior to entering the process of this invention. Front-end rectification may be desired where the ATC product stream comprises significant quantities of hydrocarbons having 3 or more carbon atoms and it is desired to remove some of the heavier of these hydrocarbons. For example, it may be desired to remove C3 and heavier or C4 and heavier hydrocarbons from the ATC product stream through the use of a front-end deethanizer or front-end depropanizer, respectively. Generally, the ATC product stream (or a portion thereof), after treatment to remove carbon dioxide and/or water, may be chilled and passed to the front-end rectification column, wherein the heavier components, for example the C3 and heavier or the C4 and heavier hydrocarbons, would be removed as a bottoms stream. The bottoms stream can be separately treated as desired.

The remaining components of the ATC product stream would be recovered as the overhead stream from the front-end rectification column and at least a portion of this overhead stream, containing a majority of the hydrogen and CO that enters the front-end rectification column, would then be passed as a mixed stream for treatment as in the first aspect of the present invention, as described above.

The ATC product stream (or a portion thereof) may need to be compressed before the at least a portion of it is passed as the mixed stream comprising carbon monoxide, hydrogen, methane and hydrocarbons with 2 or more carbons atoms to the process of the first aspect of the present invention. Compression may be performed in a suitable compressor at any convenient stage of the process.

The present invention will now be illustrated with respect to the Figures and examples, wherein:

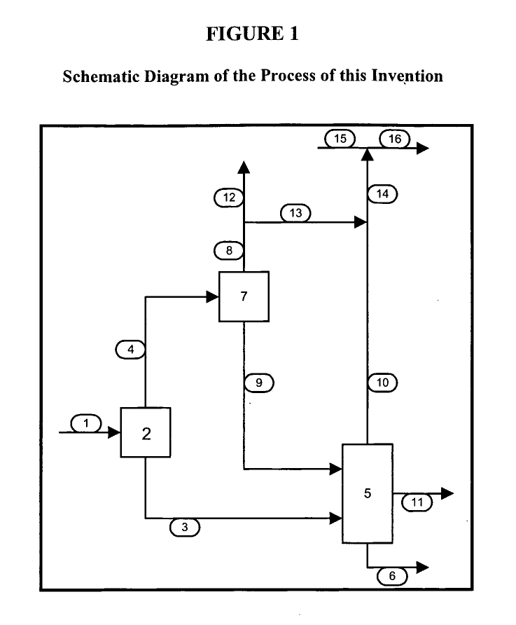

FIG. 1 depicts a schematic diagram of the process of this invention. It includes the option for combining the CO-rich stream with an externally-generated synthesis gas stream.

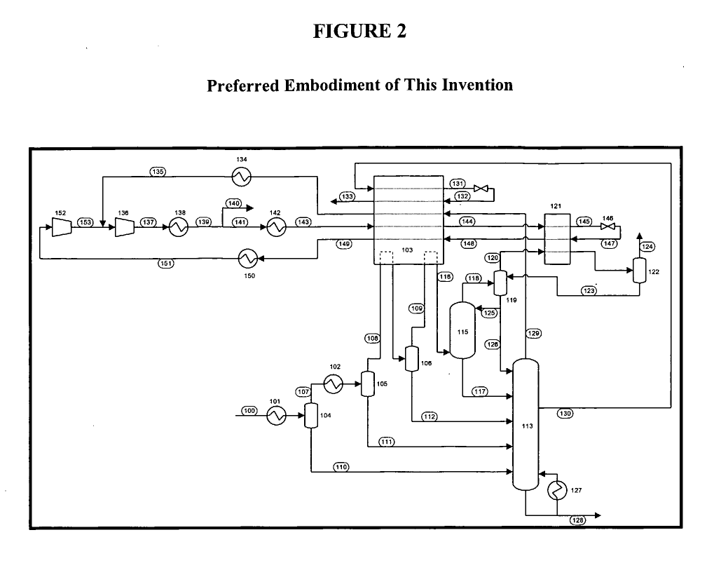

FIG. 2 depicts a more detailed implementation of the process of this invention for the simultaneous production of a hydrogen stream, a CO-rich stream, and a heavy hydrocarbon stream from the effluent of an autothermal cracking reactor. It includes an integrated CO/demethanizer column.

FIG. 1 depicts a schematic diagram of the process of this invention, one that can also produce a purified hydrogen product and a heavy hydrocarbon product.

A mixed gas stream 1 enters the process. Stream 1 comprises hydrogen, carbon monoxide, methane and hydrocarbons with 2 or more carbon atoms. It is, for example, derived from the effluent of an autothermal cracking reactor from which the carbon dioxide and water have been removed. In this case the hydrocarbons with 2 or more carbon atoms would include ethylene. The mixed gas stream is separated in step 2 into one or more first intermediate streams (3) and a second intermediate stream (4) containing a reduced concentration of hydrocarbons with 2 or more carbon atoms. Note that stream 3 is depicted in FIG. 1 as a single stream, and it is understood that it could represent a plurality of separate streams each of which are produced by separation step 2.

The one or more first intermediate streams (3) will each typically comprise at least 5 mol % hydrocarbons with 2 or more carbon atoms. The stream (3) enter separation step 5, one of the functions of which is to separate the hydrocarbons with 2 or more carbon atoms from methane and lighter components. The hydrocarbons with 2 or more carbon atoms exit in stream 6. Stream 6 typically contains few if any components as light as or lighter than methane.

Stream 4 contains hydrogen, carbon monoxide, and methane, and a relatively low concentration of hydrocarbons with 2 or more carbon atoms. It enters separation step 7 which separates the methane and carbon monoxide from the hydrogen in stream 4. Stream 8 contains primarily hydrogen and constitutes a purified hydrogen stream. Stream 9 contains primarily carbon monoxide and methane, along with smaller amounts of hydrocarbons with 2 or more carbon atoms and dissolved hydrogen. Typically stream 9 will contain less than 5 mol % hydrocarbons with 2 or more carbon atoms.

Stream 9 enters separation step 5 along with stream 3. A further function of separation step 5 is to separate methane from the carbon monoxide and hydrogen present in the feed streams to step 5. The CO-rich product stream 10 comprises primarily carbon monoxide and relatively lower concentrations of hydrogen and methane. Stream 11 comprises primarily methane and relatively low concentrations of carbon monoxide and hydrocarbons with 2 or more carbon atoms.

It should be noted that streams 3 and 9 are shown in FIG. 1 as single streams. In practice both streams 3 and 9 may represent a plurality of actual streams, for example each or both may represent multiple liquid streams resulting from multiple chilling and partial condensation and/or rectification operations within steps 2 and/or 7.

It is a feature of this invention that the stream or streams represented by streams 3 and 9 are not subjected to hydrogen stripping or to any steps directed to separation of hydrogen from the carbon monoxide existing in these streams. Both of these streams will contain a small but significant concentration of hydrogen. All of the hydrogen contained in streams 3 and 9 therefore enters the separation step 5. The lack of hydrogen separation steps is beneficial in that it reduces the amount of carbon monoxide lost to the hydrogen product or vent streams and reduces the equipment count and therefore the capital cost of the process of this invention.

If desired, the purified hydrogen stream 8 can be split. Some of the hydrogen, stream 12, can be withdrawn as a purified hydrogen product. The remainder (13) can be mixed with the CO-rich stream 10 to form a synthesis gas product stream 14. If desired, this synthesis gas product stream 14 can be mixed with a synthesis gas stream 15 from an external source. This could be done, for example, in order to control the hydrogen to carbon monoxide molar ratio (H2/CO ratio) of the final synthesis gas stream 16. Alternatively (but not shown in FIG. 1), the CO-rich stream 10 could be mixed with the external synthesis gas stream 15 to achieve a similar effect.

The CO-rich product stream 10 produced by the process of this invention will have a relatively low H2/CO molar ratio, typically significantly lower than 1.0. Externally-generated synthesis gas streams, such as stream 15 of FIG. 1, typically have higher H2/CO molar ratio, for example higher than 1.0 and often higher than 2.0. The final synthesis gas stream 16 of FIG. 1 would have an H2/CO molar ratio that is intermediate between that of the CO-rich stream and that of the externally-generated synthesis gas stream.

Tailoring of the H2/CO molar ratio of synthesis gas streams is common practice in the industrial production of synthesis gas, and has been found to be beneficial to downstream processes which utilize the combined synthesis gas stream and which operate most efficiently using synthesis gas with a certain H2/CO molar ratio. This tailoring of the H2/CO molar ratio is typically done through use of a water-gas shift reaction step which employs a reactor, catalyst, and heat transfer equipment. The use of the CO-rich stream of this invention can provide a simpler and less costly method of tailoring the H2/CO molar ratio of a synthesis gas stream.

FIG. 2 depicts a more detailed implementation of the process of this invention. All major separation, heating, and cooling steps have been shown. Some details of the process design that are well know to those skilled in the art, such as some vapor-liquid separation drums, process control valves, pumps and the like have been omitted from the drawing in order to demonstrate more clearly the key concepts of the invention. It should be noted that for clarity some heat exchangers in this detailed description are shown as individual exchangers. In practice they may be combined into one or more multi-pass cryogenic heat exchangers, as is well known to those skilled in the art.

To aid the reader in understanding this more detailed implementation, streams 110-112 in FIG. 2 correspond to the first intermediate streams (stream 3 of FIG. 1), stream 109 in FIG. 2 corresponds to the second intermediate stream (stream 4 of FIG. 1), and streams 117 and 126 of FIG. 2 correspond to the third intermediate streams (stream 9 of FIG. 1). Therefore, separation step 2 of FIG. 1 comprises drums 104-106 in FIG. 2, separation step 7 in FIG. 1 comprises units 115 and 119 in FIG. 2, and separation step 5 of FIG. 1 comprises unit 113 in FIG. 2.

A mixed gas stream 100 enters the process. Stream 100 comprises hydrogen, carbon monoxide, methane, and hydrocarbons with 2 or more carbon atoms. It is, for example derived from the effluent of an autothermal cracking reactor from which the carbon dioxide and water have been removed. In this case the hydrocarbons with 2 or more carbon atoms would include ethylene. Stream 100 is progressively cooled and partially condensed in exchangers 101, 102 and 103. After each cooling step the vapor and liquid are separated in drums 104, 105 and 106 to produce vapor streams 107, 108 and 109 and liquid streams 110, 111, and 112 as shown. The cooling in exchangers 101-103 is typically provided through cold process streams, external refrigeration, or a combination of the two. The chilling and vapor/liquid separation steps are typically carried out at relatively high pressure (e.g. typically above about 15 bar). It should be noted that more or fewer chilling stages could be used.

Liquid streams 110-112 are directed to a lower portion of column 113 and would typically enter the column at successively higher locations. If column 113 is operated at a significantly lower pressure than drums 104-106, the pressure of streams 110-112 could be reduced through the use of valves (not shown). The vapor stream 109 comprises hydrogen, carbon monoxide and methane and will typically contain a relatively low level of hydrocarbons with 2 or more carbon atoms, for example less than 5 mol % and preferably less than 1 mol % components heavier than methane. It is further chilled in a second pass through exchanger 103 and enters the bottom of the CO rectification column 115 as stream 116. Alternatively, the vapor and liquid in stream 116 could be separated and only the vapor fraction of stream 116 sent to 115. In 115 the upflowing vapor is contacted with downflowing reflux liquid, so that the gross overhead vapor from 115, stream 118, contains primarily hydrogen and CO and relatively little methane.

FIG. 2 includes an optional reflux re-contacting operation on the CO rectifier overhead stream. Accordingly, stream 118 is directed to reflux re-contacting drum 119. The overhead vapor of 119, stream 120, is chilled and partially condensed in exchanger 121. The vapor and liquid in the resulting stream are separated in reflux drum 122. The liquid from 122 is directed back to the reflux re-contacting drum 119 as stream 123. This reflux re-contacting step within drum 119 serves to chill and partially rectify the overhead vapor stream 118 while warming the reflux liquid stream 123 and partially stripping it of light material. This re-contacting step is optional, and results in overall lower energy requirement for the process. It will be apparent to those skilled in the art that in practice drum 119 and column 115 can be combined into a single vessel, and that there may be benefit to having some vapor/liquid contacting stages within 119. In such a case stream 126 would be a liquid product drawn off of the first tray, or one of the top few trays, of 115. The two vessels are depicted in FIG. 2 as separate vessels to more easily show the concept of the reflux re-contacting operation.

The vapor stream 124 from 122 constitutes the net overhead vapor of the CO rectification column 115. Stream 124 consists primarily of hydrogen with a small amount of carbon monoxide. It corresponds to stream 8 of FIG. 1 and can be treated in the same way. For example, all or portions of it could be re-warmed and withdrawn as a purified hydrogen product, expanded to a lower pressure and reheated to provide refrigeration to the process, or mixed with the CO-rich stream to increase the hydrogen to methane molar ratio of the combined stream.

The bottoms liquid stream from drum 119 comprises carbon monoxide with some dissolved hydrogen. A portion (125) is used to reflux the CO rectification column 115, and another portion is directed as reflux liquid stream 126 to the top of 113.

Alternately, column 115 could be operated as a liquid methane absorber wherein a portion of the liquid methane stream 130 (described below) would be directed as absorbent liquid to the top of column 115. In this case drums 119 and 122 and related streams would be eliminated, stream 125 would constitute the liquid methane absorbent, and stream 118 would constitute the hydrogen product stream.

Column 113 serves the function of separation step 5 of FIG. 1 and acts as a combined demethanizer/CO purification column. It is reboiled with exchanger 127. Column 113 will typically operate at a lower pressure than the chilling train vessels 104-106, 115 and 119. Three streams are produced by the column. The bottoms stream 128 contains primarily hydrocarbons with 2 or more carbon atoms and is substantially free of methane and lighter components, for example containing less than 0.1 mol % methane and lighter components. A CO-rich overhead stream 129 is withdrawn from 113 and contains primarily carbon monoxide and a lower concentration of hydrogen, for example less than 20 mol % but typically more than about 2 mol % H2. A liquid sidestream 130 is taken from column 113 at an intermediate location. This stream contains primarily methane with dissolved hydrogen and a relatively low concentration of hydrocarbons with 2 or more carbon atoms, for example less than 5 mol %, and a relatively low concentration of carbon monoxide, for example less than 10 mol %.

Column 113 can be described as consisting of three sections, each of which has a distinct separation function. In the upper section, between the overhead vapor product stream 129 and the sidestream 130, is the CO purification section in which methane is separated from CO and hydrogen so that a CO-rich stream is recovered as the column overhead stream 129 and a methane-rich stream is recovered as the sidestream 130. The stream or streams that enter the CO purification section (streams 117 and 126 in FIG. 2) correspond to stream 9 of FIG. 1 and will typically contain less than 5 mol % hydrocarbons with 2 or more carbon atoms. The middle section, between the sidestream 130 and the lowest feed stream 110, constitutes a demethanizer rectification section in which hydrocarbons with 2 or more carbon atoms (principally ethylene) are recovered from the methane and lighter components. The stream or streams that enter the demethanizer rectification section (streams 110-112 in FIG. 2) correspond to stream 3 of FIG. 1 and will typically contain at least 5 mol % hydrocarbons with 2 or more carbon atoms. The lower section, below the lowest feed stream 110, constitutes the demethanizer stripping section in which methane and lighter components are stripped from the hydrocarbons with 2 or more carbon atoms, which exit in bottoms stream 128 as described above.

A further feature of the process of this invention is that the CO purification combined column 113 can operate at relatively higher pressures than CO purification columns of the prior art. For example, U.S. Pat. No. 5,351,491 teaches operating the CO purification column at a pressure of around 5.9 bara and U.S. Pat. No. 6,578,377 teaches operating the CO purification column at a pressure of around 2.8 bara. In contrast, the combined column 113 of this invention can operate efficiently at pressures above 7 bara, and preferably operates at pressures above 10 bara. Operation of the CO purification column of the present invention at relatively higher pressures than the prior art processes provides energy benefits. For example, reflux liquid can be provided to the column at relatively higher temperatures than prior-art processes. In addition, the energy required to compress the overhead product stream 129 is lower than in prior art processes, reducing the energy demand of the product compressors or a CO refrigeration compressor, if used.

The ability of the CO purification column of the present invention to efficiently operate at higher pressures is a direct result of the elimination of the hydrogen stripping steps from streams 110-112 and 117. It is also enhanced by combining the demethanizer and CO purification columns so that stripping vapor to the CO purification section is supplied directly by vapors from the demethanizer rectification section, rather than through the use of a conventional reboiler as in the prior art.

As described, sidestream 130 is taken from a point on the column where there is a suitably low concentration of hydrocarbons with 2 or more carbon atoms and carbon monoxide in the liquid. It is optionally pumped to a higher pressure and subcooled in exchanger 103 as shown. Typically this stream will be cooled in 103 to at least 5° C. below its bubble point. The bubble point is defined as that temperature at which the first bubble of vapor forms in the liquid of stream 130, at the pressure in column 113 at the point from which stream 130 is withdrawn. The subcooled stream 131 can be expanded to a lower pressure stream 132 and vaporized to provide refrigeration in exchanger 103. The warmed stream 133 can be further warmed elsewhere in the system and would typically be used as fuel. The expanded stream 132 can advantageously be combined with other streams with lower molecular weight, for example an expanded portion of the hydrogen stream 124 (not shown), to reduce the temperature at which the combined stream is vaporized in exchanger 103.

Refrigeration to the process of FIG. 2 is at least partially provided by a recompression refrigeration system which uses the CO-rich product vapor as at least a portion of the working fluid. A simple example showing the key elements of such a system is depicted within FIG. 2. Those skilled in the art will recognize that many design options exist for such a system and the simple system depicted in FIG. 2 is not meant to preclude any of the potential design options that could be applied to the CO-rich refrigeration system.

The CO-rich product stream 129 is warmed in exchanger 103 and optionally in another exchanger 134 and the resulting stream 135 is compressed in 136 to a suitable pressure, for example about 20 barg. If the compressed stream 137 is significantly above ambient temperature it can be cooled to near ambient temperature in exchanger 138. The resulting high-pressure CO-rich stream 139 is split into two streams. Stream 140 is the net CO-rich stream from the process and corresponds to the CO-rich product stream 10 of FIG. 1. It can be recovered as the final CO-rich product or can be further treated in various ways.

These further treatment options are not shown on FIG. 2, but include, for example, production of a purified carbon monoxide stream from stream 140. This can be accomplished, for example, by removing the hydrogen contained in stream 140 through the use of the methanation reaction

CO+3H₂->CH₄+H₂O

in contact with a suitable catalyst such as nickel catalyst or other suitable material under methanation conditions to produce an essentially hydrogen-free CO product stream. Alternatively, the hydrogen could be adsorbed on a suitable material such as natural or synthetic zeolite or other suitable material such that an essentially hydrogen-free CO product stream is produced. The hydrogen-containing adsorbent would then typically be regenerated by changing the pressure or temperature of the adsorbent or through other suitable means well known to those skilled in the art. Such a system is a significant departure from the prior art of U.S. Pat. No. 5,351,491, which teaches a process which produces a purified hydrogen stream rather than the purified CO stream of this invention from a mixed hydrogen/CO stream.

Alternatively stream 140 could be combined with all or a portion of the hydrogen in stream 124 to produce a synthesis gas product with a higher hydrogen to carbon monoxide ratio than exists in stream 140. As described in FIG. 1, stream 140 could also be combined with an externally-generated synthesis gas stream as a means of tailoring the hydrogen to carbon monoxide ratio of the combined syngas stream. These and other potential uses for stream 140 are all contained within the scope of this invention.

Stream 141 is further cooled in exchanger 142 against cold process streams or an external refrigeration source. In practice exchanger 142 will typically consist of multiple exchangers or one or more multi-pass cryogenic exchangers as are well-known to those skilled in the art. The cooled stream 143 enters exchanger 103 where it is at least partially condensed to form stream 144. It is a feature of this invention that at least a portion of the refrigeration required for condensing stream 143 can be derived from the vaporization of at least a portion of the methane-rich stream 130. Stream 144 is further cooled in exchanger 121 to produce stream 145. The pressure of stream 145 is reduced through valve 146 and the resulting low-pressure stream 147 is partially vaporized in exchanger 121 to provide refrigeration for refluxing the CO rectification column 115 as shown. The partially vaporized stream 148 is further heated to provide refrigeration to exchanger 103, thereby producing warmed stream 149. Stream 149 can be further reheated in exchanger 150 if desired. The final low-pressure reheated stream 151 is directed to compressor 152. In 152 the low-pressure stream is recompressed in one or more stages and optionally cooled to produce an intermediate-pressure stream 153. This stream is combined with stream 135 and the combined stream directed to compressor 136 as shown.

### EXAMPLE 1

This is an example of the process of the present invention for use in the simultaneous recovery of a CO-rich product, a purified hydrogen product, and a heavy hydrocarbon product from a mixed hydrocarbon stream derived from the effluent of an autothermal cracking reactor. The recovery process of this example was simulated using a commercially available process simulation package. The process simulated in the example is based on the embodiment of FIG. 2. In the example the subcooled methane product stream 132 is combined with an expanded and re-warmed portion of the hydrogen product stream 124 (not shown). Selected stream information is given in Table 1, with stream numbers referenced to FIG. 2. Exchanger duties for the example (in MW) are given in Table 2. For exchangers 103 and 121 the value in Table 2 corresponds to the net chilling duty delivered to the process stream being chilled.

TABLE 1
Flows and Conditions for Streams in Example 1 (FIG. 2)

| Stream No. | 100 | 107 | 108 | 109 | 110 | 111 | 112 | 117 | 118 | 124 |
|---|---|---|---|---|---|---|---|---|---|---|
| Temperature (Deg C.) | −26.9 | −70.0 | −110.0 | −138.0 | −40.0 | −110.0 | −138.0 | −171.2 | −176.8 | −193.0 |
| Pressure (barg) | 25 | 25 | 25 | 25 | 25 | 25 | 25 | 24 | 24 | 24 |
| Vapor Fraction | 0.93 | 1.00 | 1.00 | 1.00 | 0.00 | 0.00 | 0.00 | 0.00 | 0.00 | 1.00 |

Molar flows (kg mol/hr)

| | | | | | | | | | | |
|---|---|---|---|---|---|---|---|---|---|---|
| HYDROGEN | 3976.1 | 3908.9 | 3891.4 | 3886.1 | 67.2 | 17.5 | 5.3 | 41.9 | 3860.6 | 3821.4 |
| CO | 1737.1 | 1612.9 | 1531.8 | 1435.4 | 124.2 | 81.2 | 46.4 | 728.5 | 1131.9 | 234.8 |
| METHANE | 1898.2 | 1479.4 | 1141.0 | 915.9 | 418.9 | 338.4 | 225.0 | 912.1 | 6.6 | 0.0 |
| ETHYLENE | 3885.9 | 1054.1 | 149.7 | 14.5 | 2831.8 | 904.4 | 135.2 | 14.5 | 0.0 | 0.0 |
| ETHANE | 3392.8 | 505.4 | 33.0 | 1.0 | 2887.4 | 472.5 | 31.9 | 1.0 | 0.0 | 0.0 |
| ACETYLEN | 36.0 | 6.3 | 0.5 | 0.0 | 29.7 | 5.8 | 0.5 | 0.0 | 0.0 | 0.0 |
| PROPYLEN | 244.7 | 3.6 | 0.0 | 0.0 | 241.1 | 3.6 | 0.0 | 0.0 | 0.0 | 0.0 |
| PROPANE | 53.0 | 0.5 | 0.0 | 0.0 | 52.6 | 0.5 | 0.0 | 0.0 | 0.0 | 0.0 |
| PROPDIEN | 5.9 | 0.0 | 0.0 | 0.0 | 5.9 | 0.0 | 0.0 | 0.0 | 0.0 | 0.0 |
| C4+ | 267.8 | 0.1 | 0.0 | 0.0 | 267.7 | 0.1 | 0.0 | 0.0 | 0.0 | 0.0 |

| Stream No. | 126 | 128 | 129 | 130 | 140 | 145 | 148 | 151 | 153 |
|---|---|---|---|---|---|---|---|---|---|
| Temperature (Deg C.) | −178.2 | −30.2 | −161.2 | −122.6 | 40.0 | −193.0 | −179.8 | −46.3 | 40.0 |
| Pressure (barg) | 24 | 13 | 13 | 13 | 25 | 24 | 1 | 1 | 12 |
| Vapor Fraction | 0.00 | 0.00 | 1.00 | 0.00 | 1.00 | 0.06 | 1.00 | 1.00 | 1.00 |

Molar flows (kg mol/hr)

| | | | | | | | | | |
|---|---|---|---|---|---|---|---|---|---|
| HYDROGEN | 22.8 | 0.0 | 150.4 | 2.9 | 152.3 | 59.1 | 59.7 | 59.7 | 59.7 |
| CO | 522.1 | 0.0 | 1392.9 | 77.1 | 1518.0 | 603.2 | 695.9 | 695.9 | 695.9 |
| METHANE | 3.8 | 1.6 | 6.3 | 1890.1 | 6.5 | 2.7 | 2.7 | 2.7 | 2.7 |
| ETHYLENE | 0.0 | 3872.3 | 0.0 | 13.6 | 0.0 | 0.0 | 0.0 | 0.0 | 0.0 |
| ETHANE | 0.0 | 3391.9 | 0.0 | 0.9 | 0.0 | 0.0 | 0.0 | 0.0 | 0.0 |
| ACETYLEN | 0.0 | 36.0 | 0.0 | 0.0 | 0.0 | 0.0 | 0.0 | 0.0 | 0.0 |
| PROPYLEN | 0.0 | 244.7 | 0.0 | 0.0 | 0.0 | 0.0 | 0.0 | 0.0 | 0.0 |
| PROPANE | 0.0 | 53.0 | 0.0 | 0.0 | 0.0 | 0.0 | 0.0 | 0.0 | 0.0 |
| PROPDIEN | 0.0 | 5.9 | 0.0 | 0.0 | 0.0 | 0.0 | 0.0 | 0.0 | 0.0 |
| C4+ | 0.0 | 267.8 | 0.0 | 0.0 | 0.0 | 0.0 | 0.0 | 0.0 | 0.0 |

TABLE 2
Heat Exchanger Duties

| Exchanger | Net Duty (MW) |
|---|---|
| 101 | −23.88 |
| 102 | −8.77 |
| 103 | −7.22 |
| 121 | −1.77 |
| 127 | 8.38 |
| 138 | −2.50 |
| 142 | −1.28 |
| 150 | 0.52 |

1. A process for the recovery of a CO-rich stream comprising at least 50 mol % carbon monoxide and at least 2 mol % hydrogen from a mixed stream comprising carbon monoxide, hydrogen, methane and hydrocarbons with two or more carbon atoms, said process comprising the steps of:
i. passing the mixed stream to a first separation step to separate the mixed stream into one or more first intermediate streams comprising hydrocarbons with two or more carbon atoms and a reduced quantity of the carbon monoxide, hydrogen and methane relative to the mixed stream, and a second intermediate stream comprising carbon monoxide, hydrogen, methane and a reduced quantity of the hydrocarbons with 2 or more carbons atoms relative to the mixed stream,
ii. passing the second intermediate stream to a second separation step to separate the second intermediate stream into one or more third intermediate streams comprising carbon monoxide and methane and a reduced quantity of hydrogen relative to the second intermediate stream, and a hydrogen-rich stream which comprises a reduced quantity of carbon monoxide and methane relative to the second intermediate stream, and
iii. passing at least one of the one or more first intermediate streams and at least one of the one or more third intermediate streams to a third separation step to recover a CO-rich stream comprising at least 50 mol % carbon monoxide and at least 2 mol % hydrogen, a methane-rich stream and a heavy hydrocarbon stream comprising hydrocarbons with 2 or more carbon atoms,
wherein the at least one of the one or more first intermediate streams and the at least one of the one or more third intermediate streams are passed to the third separation step without treatment to separate hydrogen.

2. The process of claim 1 wherein the third separation step comprises a single distillation column which acts as a combined demethanizer/CO purification column, and from which is removed a gaseous overhead stream, a liquid sidestream, and a liquid bottoms stream, as respectively said CO-rich stream, said methane-rich stream and said heavy hydrocarbon stream.

3. The process of claim 1 wherein the CO-rich stream comprises at least 80 mol % carbon monoxide and at least 5 mol % hydrogen.

4. The process of claim 2 wherein each of the one or more third intermediate streams enters the single distillation column at a location above that where the liquid sidestream is recovered, and each of the one or more first intermediate streams enters the single distillation column at a location below that where the liquid sidestream is recovered.

5. The process of claim 2 wherein the top pressure of said single distillation column is above 7 bara.

6. The process of claim 5 wherein the top pressure of said single distillation column is above 1.0 bara.

7. The process of claim 1 wherein the first separation step comprises passing the mixed stream to a chilling train comprising one or more stages of partial condensation of the process stream.

8. The process of claim 1 wherein the second separation step comprises at least one step of chilling and partial condensation of the second intermediate stream.

9. The process of claim 8 wherein the second separation step comprises at least one step of rectification of the second intermediate stream.

10. The process of claim 1 in which the hydrogen in said CO-rich stream is removed through adsorption onto a suitable adsorbent or through reaction with the carbon monoxide contained in said CO-rich stream to produce a purified carbon monoxide stream containing less than 1 mol % hydrogen.

11. The process of claim 1 wherein at least a portion of said CO-rich stream is used as at least a portion of the working fluid in a CO-rich vapor recompression refrigeration system, the CO-rich vapor recompression refrigeration system providing process chilling duty to one or more of the first, second, and/or third separation steps.

12. The process of claim 11 in wherein said CO-rich vapor recompression refrigeration system comprises the steps of:
i. Compressing at least a portion of said CO-rich stream to a relatively high pressure to form a relatively high-pressure CO-rich stream,
ii. Cooling and partially condensing at least a portion of the relatively high-pressure CO-rich stream of step (i) to produce a cooled and at least partially condensed working fluid stream,
iii. Reducing the pressure of the cooled and at least partially condensed working fluid stream of step (ii) to produce a reduced-pressure working fluid stream,
iv. Heating and at least partially vaporizing the reduced-pressure working fluid stream of step (iii) to provide the process chilling duty to one or more of the first, second, and/or third separation steps and produce a heated reduced pressure working fluid stream, and
v. Recompressing at least a portion of the heated reduced pressure working fluid stream in step (i)

13. The process of claim 12 in which the methane-rich stream is recovered as a liquid stream and at least a portion of said methane-rich stream is reduced in pressure, warmed, and at least partially vaporized to provide at least a portion of the cooling duty needed in step (ii).

14. The process of claim 13 wherein said methane-rich stream is optionally pumped and then cooled to a temperature at least 5° C. below its bubble point before it is reduced in pressure, warmed, and at least partially vaporized, said bubble point being defined at the pressure at which said methane-rich stream is recovered and prior to the optional pumping step.

15. The process of claim 13 wherein the at least a portion of said methane-rich stream is first mixed with at least a portion of said hydrogen-rich stream and the combined stream is warmed and at least partially vaporized to provide at least a portion of the cooling duty needed in step (ii).

16. The process of claim 1 wherein the methane-rich stream contains less than 5 mol % hydrocarbons with 2 or more carbon atoms.

17. The process of claim 1 wherein the concentration of hydrogen in the hydrogen-rich stream is at least 80 mol %.

18. The process of claim 1 wherein the concentration of hydrogen in the CO-rich stream is no more than 20 mol %.

19. The process of claim 1 wherein at least a portion of the CO-rich stream is mixed with an externally-generated synthesis gas stream which has a different molar hydrogen to carbon monoxide ratio than the CO-rich stream to produce a combined synthesis gas stream which has a molar hydrogen to carbon monoxide ratio which is different from that of either the CO-rich stream or the externally-generated synthesis gas stream.
